(19) United States
(12) Patent Application Publication
(10) Pub. No.: US 2020/0325522 A1
(43) Pub. Date: Oct. 15, 2020

(54) METHOD AND SYSTEMS TO CHARACTERIZE TUMORS AND IDENTIFY TUMOR HETEROGENEITY

(71) Applicant: Mission Bio, Inc., South San Francisco, CA (US)

(72) Inventors: Pedro Mendez, San Mateo, CA (US); Dalia Dhingra, San Francisco, CA (US); David Ruff, San Francisco, CA (US); Aik Ooi, San Mateo, CA (US)

(73) Assignee: Mission Bio, Inc., South San Francisco, CA (US)

(21) Appl. No.: 16/839,058

(22) Filed: Apr. 2, 2020

Related U.S. Application Data
(60) Provisional application No. 62/828,416, filed on Apr. 2, 2019.
(60) Provisional application No. 62/828,386, filed on Apr. 2, 2019.
(60) Provisional application No. 62/828,420, filed on Apr. 2, 2019.
(60) Provisional application No. 62/829,358, filed on Apr. 4, 2019.

(51) Int. Cl. C12Q 1/6804; C12N 15/10; C12Q 1/6886; G01N 33/574

(52) U.S. Cl. CPC C12Q 1/6804; C12N 15/1096; C12Q 1/6886; C12N 15/1075; C12Q 2600/158; C12Q 2600/156; G01N 2458/10; G01N 2570/00; G01N 33/57484

(57) ABSTRACT

Provided herein are methods for detection and characterization of a target nucleic acid from a single cell. Some embodiments highlight the capability of identifying the biologically relevant variants at the moment of the diagnosis, but also how the treatment positively select resistant cellular clones based on the mutation signature. This positions the Tapestri™ platform described herein as the only tool available to study how genetic variants co-exist and which combinations are sensitive and resistant to certain treatments. Thus, it helps in the diagnostic precision, treatment follow up and new target identification and drug development

### FIELD

This invention relates generally to the detection and identification of target nucleic acids and mutations and allelic variants in a target nucleic acid, and more particularly to the detection and identification of target nucleic acids and mutations and allelic variants in a target nucleic acid in a single cell.

### RELATED APPLICATIONS

This application takes priority to the following U.S. Provisional Application Ser. No. 62/828,416 filed Apr. 2, 2019 and entitled ‘Analytical Methods To Identify Tumor Heteregeneity’; U.S. Ser. No. 62/829,291 filed Apr. 4, 2019 and entitled ‘Method, System And Apparatus For Antibody Tag Priming And Genomic Dna Bridge’; U.S. Ser. No. 62/828,386 filed Apr. 2, 2019 and entitled ‘A Complete Solution For Height Throughput Single Cell Sequencing; U.S. Ser. No. 62/828,420 filed Apr. 2, 2019 and entitled ‘Method and Apparatus for Universal base library preparation’; and U.S. Ser. No. 62/829,358 filed Apr. 4, 2019 and entitled ‘Method and Apparatus for Fusion in DNA and RNA’, all incorporated by reference herein.

### BACKGROUND

There is a need for method, system and apparatus to provide high-throughput, single-cell nucleic acid detection and characterization. There is also need for method, system and apparatus to provide high-throughput, single-cell analyte detection and analysis that includes the detection and identification of target nucleic acids and mutations and allelic variants in a target nucleic acid.

With the advancements of single cell sequencing technologies, it is now possible to interrogate thousands of cells in a single experiment. Single-cell RNA-Seq has been available for several years but high-throughput single-cell DNA analysis is in its infancy. In order to perform next generation Proteomic and Genomic analysis and mapping, it is essential to develop new capabilities for assessing genetic variation present in rare cells and to better understand the role that these cells play in the evolution of tumor progression. If these challenges are addressed, new opportunities to develop novel approaches to map and identify genetic diversity in cancer cell populations exist. With this, new opportunities for monitoring and treatment of cancer and other diseases also become possible.

Proteins are the primary effectors of cellular function, including cellular metabolism, structural dynamics, and information processing. Proteins are the physical building blocks of cells, comprising the majority of cell mass and carrying out most cell functions, including cell structure dynamics, metabolism, and information processing. They are the molecular machines that convert thermodynamic potential into the energy of living systems. Measuring protein expression and modification is thus important for obtaining an accurate snapshot of cell state and function. A common challenge when measuring proteins at the single-cell level is that most cell systems are heterogeneous, containing massive numbers of molecularly distinct cells. A centimeter-sized tissue volume, for example, can contain billions of cells, each with its own unique spectrum of protein expression and modification; moreover, this underlying cellular heterogeneity can have important consequences on the system as a whole, such as in development, the regulation of the immune system, cancer progression and therapeutic response. For heterogeneous systems like these, methods for high-throughput protein profiling in single cells are necessary.

Profiling proteins in single cells at high throughput requires methods that are sensitive and fast. Flow cytometry with fluorescently-labeled antibodies has been a bedrock in biology for decades because it can sensitively profile proteins in millions of single cells. By labeling antibodies with dyes of different color, profiling can be multiplexed to tens of proteins. By swapping dyes with mass tags and using a mass spectrometer for the readout, multiplexing can be increased to over a hundred antibodies. Nevertheless, while these methods continue to improve in sensitivity and multiplexing, they remain far from enabling the characterization of the entire proteome in single cells, which for humans comprises >20,000 proteins and >100,000 epitopes. A system that could sensitively profile all epitopes in a proteome would be extremely valuable, because it would obviate the need to select which proteins to target. However, existing methods with dye and mass tags are not scalable to the level of full proteome analysis, and in the case of mass-cytometry, destroy the transcriptome during analysis, making it challenging to obtain simultaneous measurements of proteome and transcriptome from the same single cell. (see Shahi, P., Kim, S., Haliburton, J. et al. Abseq: Ultrahigh-throughput single cell protein profiling with droplet microfluidic barcoding. Sci Rep 7, 44447 (2017). https://doi.org/10.1038/srep44447).

The inventions described herein meet these unsolved challenges and needs. To address these challenges and enable the characterization of genetic diversity in cancer cell populations, we developed a novel approach to identify mutation signatures which define subclones present in a tumor population.

### BRIEF SUMMARY

The inventions described and claimed herein have many attributes and embodiments including, but not limited to, those set forth or described or referenced in this Brief Summary. The inventions described and claimed herein are not limited to, or by, the features or embodiments identified in this Summary, which is included for purposes of illustration only and not restriction.

In a first aspect, embodiments of the invention are directed to methods of identifying and characterizing clonal sub populations of cells, where an exemplary non-limiting method of the invention includes, independent of order, the steps of: conjugating barcode sequences flanked by PCR priming sites onto antibodies, where a barcode sequence is specific to an antibody; performing a cell staining step using the barcode-conjugated antibodies; partitioning or separating individual cells or nuclei, and encapsulating one or more individual cell(s) or nuclei, in a reaction mixture including a protease and/or reverse transcriptase; incubating the encapsulated cell with the protease in the drop to produce cDNA in a cell lysate with released chromatin; providing one or more nucleic acid amplification primer sets, wherein one or more primer of a primer set has a barcode identification sequence associated with an antibody; performing a nucleic acid amplification reaction to produce one or more amplicons; providing an affinity reagent that includes a nucleic acid sequence complementary to the identification barcode sequence of one of more nucleic acid primer of a primer set, where the affinity reagent having a nucleic acid sequence complementary to the identification barcode sequence is capable of binding to a nucleic acid amplification primer set having a barcode identification sequence; contacting an affinity reagent to the amplification product comprising amplicons of one or more target nucleic acid sequence under conditions sufficient for binding of the affinity reagent to the target nucleic acid to form an affinity reagent bound target nucleic acid; and determining the identity and characterizing one or more protein by sequencing a barcode of an amplicon.

In some implementations of this embodiment and others, signature mutations are identified at a single-cell level.

Another exemplary non-limiting method of the invention includes, independent of order, the steps of: selecting one or more target nucleic acid sequence in an individual cell, where the target nucleic acid sequence is contained in a DNA or RNA; providing a sample having one or more individual single cell; encapsulating an individual cell in a drop; incubating the encapsulated cell in presence of protease and/or reverse transcriptase in the drop to produce cDNA and a cell lysate; providing a nucleic acid amplification primer set complementary to a target nucleic acid, where at least one primer of the nucleic acid amplification primer set comprises a barcode identification sequence; performing a reverse transcription and nucleic acid amplification reaction to form an amplification product from the nucleic acid of a single cell; and determining whether the target nucleic acid is expressed if the target nucleic acid comprises a transcript.

In some implementations of this embodiment and others, further include providing an affinity reagent that has a nucleic acid sequence complementary to a barcode sequence of one of more nucleic acid primer, where the affinity reagent having the nucleic acid sequence complementary to the barcode sequence is capable of binding to a nucleic acid amplification primer having a barcode sequence; and contacting an affinity reagent to the amplification product that includes amplicons under conditions sufficient for binding of the affinity reagent to the target nucleic acid to form an affinity reagent bound target nucleic acid.

### BRIEF DESCRIPTION OF THE DRAWINGS

FIG. 1 is a schematic illustrating the use of embodiments directed to analyzing nucleic acids or proteins from a single cell sample. This illustration shows why single cell genomic and proteomic analysis is important to study the complexity of tumor mutagenesis and cancer evolution. The Mission Bio Tapestri™ workflow illustrated is a preferred system for some embodiments because it allows tumors and cancers to be analyzed at a single cell level.

FIG. 2 is a schematic illustrating an embodiment for the primary analysis and subclone identification. A. Steps of the Tapestri™ Pipeline to preprocess, align sequencing reads and call cells from sequencing data. B. Filters applied to single-cell data generated with Tapestri™ platform, through Tapestri Insights™ software, to ensure that only complete and high-quality data is analyzed for tumor heterogeneity analysis. C. Examples of data completeness of three targeted DNA panels, AML, CLL and Myeloid, respectively.

FIG. 3 presents data relating to selecting informative variants. A. Attributes of selected variants shown in Tapestri Insights™ user interface. A likely pathogenic, missense variant in the U2AF1 gene (p.Q157P), is highlighted. The violin plot manifests distribution of the single-cell variant allele frequency of the variant across the three cell sub-populations detected. The left violin plot shows a population of cells with a median 45% of scVAF (%), characteristic of a heterozygous genotype. The middle line shows a group of cells with WT genotype; while the right violin plot shows another subpopulation of cells with homozygous genotype. B. Sample matrix containing genotype ID. Each variant is organized in different column, while each cell located in independent row. The bottom table is a screenshot of the visualization of the different cell clones (C1, WT, C3 and C4) as results of selecting two variants. Zygosity of genotypes are organized in columns. In this example the Clone C1 is homozygous for EZH2:chr7:148543621:G/A and heterozygous for U2AF1:chr21:44514777:T/G, and represents 46.32% of all cells called by the pipeline.

FIG. 4 presents sensitivity of the platform in detecting of know genotypes. Data from three independent experiments are shown. A. A mixture of four cell lines containing: 98.4% of PC3, 1% of DU145, 1% of HCT15 and 0.5% of SKEMEL28 cell lines was loaded in Tapestri™, and libraries of the AML panel were prepared sequenced. After the pipeline, previously known variants (shown in tables), were used to identify different cell types and visualized by PCA dimensional reduction method. B. The same cell mixture from panel A was used to prepare single-cell libraries using the Myeloid panel. C. Limit of detection (LOD) experiment with mixtures of Raji and K562 cell lines with 99/1%, 99.5/0.5% and 99.9/0.1%, respectively. Dimensional reduction visualization of cell populations based on selected known genotypes by PCA.

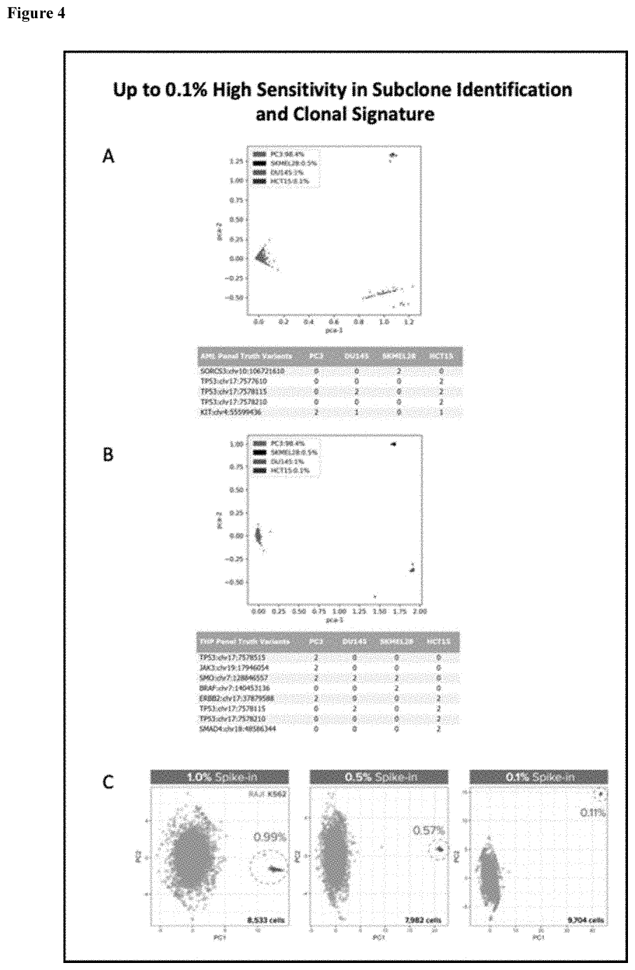

FIG. 5 presents data of longitudinal analysis, relating the clonal evolution of different samples of an AML patient, during the course of treatment. A. Fishplot and B, dotplot showing the dynamism of all the cellular subclones. The triple FLT3 clone is eliminated by the FLT3 inhibitor, however two treatment-resistant clones are expanded during the treatment. The double mutant clone (IDH2/SF3B1), that lead the treatment failure (highlighted in green). Only single-cell technology could have been accurately detected the raising double-mutant clone at days 28 and 112 after initiating the treatment with FLT3 inhibitor.

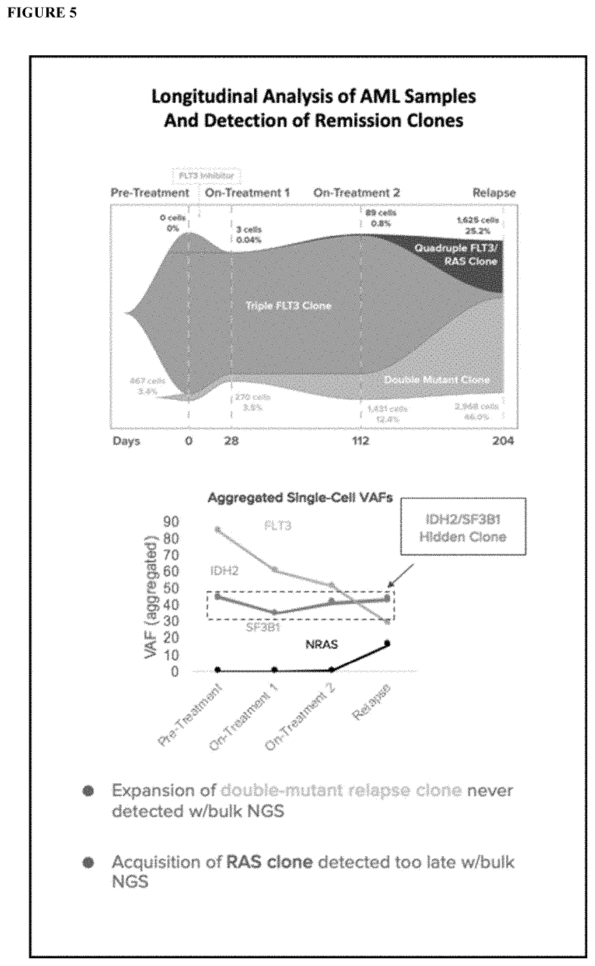

FIG. 6 is a schematic illustration of the clonal distribution and phylogeny analysis used to detect rare subclones and tumor purity. Four metastatic and one. “normal”, frozen tissues obtained during the autopsy of a metastatic melanoma patient, were used to reconstruct the phylogenetic tree. First, the nuclei were isolated from each frozen tissue, using Tapestri™ and the Tumor Hot-Spot panel. A total of 6 clinically relevant variants were detected across the five tissues analyzed. Using the theory that assumes that the number of somatic variants acquired, increases with time, we reconstructed the complete phylogenetic tree of the patient (panel A), as well as per each tissue analyzed (panel B). C. Zigosity of Co-existing variants (rows) per clone (columns). D and E. Barplots with % of each cell clone across each tissue type. The sensitivity of the single-cell technology showed in FIG. 4, is also showcased in this example, where 0.15% of the 6,400 cells sequenced from the “normal” liver tissue, were carriers of the three somatic mutations characteristic of the chest wall 1 and liver metastatic tissues, respectively. The doctors that performed the autopsy, confirmed the presence of oligometastatic disease in the “normal” liver.

### DETAILED DESCRIPTION

Various aspects of the invention will now be described with reference to the following section which will be understood to be provided by way of illustration only and not to constitute a limitation on the scope of the invention.

“Complementarity” refers to the ability of a nucleic acid to form hydrogen bond(s) or hybridize with another nucleic acid sequence by either traditional Watson-Crick or other non-traditional types. As used herein “hybridization,” refers to the binding, duplexing, or hybridizing of a molecule only to a particular nucleotide sequence under low, medium, or highly stringent conditions, including when that sequence is present in a complex mixture (e.g., total cellular) DNA or RNA. See e.g. Ausubel, et al., Current Protocols In Molecular Biology, John Wiley & Sons, New York, N.Y., 1993. If a nucleotide at a certain position of a polynucleotide is capable of forming a Watson-Crick pairing with a nucleotide at the same position in an anti-parallel DNA or RNA strand, then the polynucleotide and the DNA or RNA molecule are complementary to each other at that position. The polynucleotide and the DNA or RNA molecule are “substantially complementary” to each other when a sufficient number of corresponding positions in each molecule are occupied by nucleotides that can hybridize or anneal with each other in order to affect the desired process. A complementary sequence is a sequence capable of annealing under stringent conditions to provide a 3′-terminal serving as the origin of synthesis of complementary chain.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” and “similarity” can be readily calculated by known methods, including, but not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H G, eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., Siam J. Applied Math., 48:1073 (1988). In addition, values for percentage identity can be obtained from amino acid and nucleotide sequence alignments generated using the default settings for the AlignX component of Vector NTI Suite 8.0 (Informax, Frederick, Md.). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215:403-410 (1990)). The BLAST X program is publicly available from NCBI and other sources (BLAST Manual, Altschul, S., et al., NCBINLM NIH Bethesda, Md. 20894: Altschul, S., et al., J. Mol. Biol. 215:403-410 (1990). The well-known Smith Waterman algorithm may also be used to determine identity.

The terms “amplify”, “amplifying”, “amplification reaction” and their variants, refer generally to any action or process whereby at least a portion of a nucleic acid molecule (referred to as a template nucleic acid molecule) is replicated or copied into at least one additional nucleic acid molecule. The additional nucleic acid molecule optionally includes sequence that is substantially identical or substantially complementary to at least some portion of the template nucleic acid molecule. The template nucleic acid molecule can be single-stranded or double-stranded and the additional nucleic acid molecule can independently be single-stranded or double-stranded. In some embodiments, amplification includes a template-dependent in vitro enzyme-catalyzed reaction for the production of at least one copy of at least some portion of the nucleic acid molecule or the production of at least one copy of a nucleic acid sequence that is complementary to at least some portion of the nucleic acid molecule. Amplification optionally includes linear or exponential replication of a nucleic acid molecule. In some embodiments, such amplification is performed using isothermal conditions; in other embodiments, such amplification can include thermocycling. In some embodiments, the amplification is a multiplex amplification that includes the simultaneous amplification of a plurality of target sequences in a single amplification reaction. At least some of the target sequences can be situated, on the same nucleic acid molecule or on different target nucleic acid molecules included in the single amplification reaction. In some embodiments, “amplification” includes amplification of at least some portion of DNA- and RNA-based nucleic acids alone, or in combination. The amplification reaction can include single or double-stranded nucleic acid substrates and can further including any of the amplification processes known to one of ordinary skill in the art. In some embodiments, the amplification reaction includes polymerase chain reaction (PCR). In the present invention, the terms “synthesis” and “amplification” of nucleic acid are used. The synthesis of nucleic acid in the present invention means the elongation or extension of nucleic acid from an oligonucleotide serving as the origin of synthesis. If not only this synthesis but also the formation of other nucleic acid and the elongation or extension reaction of this formed nucleic acid occur continuously, a series of these reactions is comprehensively called amplification. The polynucleic acid produced by the amplification technology employed is generically referred to as an “amplicon” or “amplification product.”

A number of nucleic acid polymerases can be used in the amplification reactions utilized in certain embodiments provided herein, including any enzyme that can catalyze the polymerization of nucleotides (including analogs thereof) into a nucleic acid strand. Such nucleotide polymerization can occur in a template-dependent fashion. Such polymerases can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze such polymerization. Optionally, the polymerase can be a mutant polymerase comprising one or more mutations involving the replacement of one or more amino acids with other amino acids, the insertion or deletion of one or more amino acids from the polymerase, or the linkage of parts of two or more polymerases. Typically, the polymerase comprises one or more active sites at which nucleotide binding and/or catalysis of nucleotide polymerization can occur. Some exemplary polymerases include without limitation DNA polymerases and RNA polymerases. The term “polymerase” and its variants, as used herein, also includes fusion proteins comprising at least two portions linked to each other, where the first portion comprises a peptide that can catalyze the polymerization of nucleotides into a nucleic acid strand and is linked to a second portion that comprises a second polypeptide. In some embodiments, the second polypeptide can include a reporter enzyme or a processivity-enhancing domain. Optionally, the polymerase can possess 5′ exonuclease activity or terminal transferase activity. In some embodiments, the polymerase can be optionally reactivated, for example through the use of heat, chemicals or re-addition of new amounts of polymerase into a reaction mixture. In some embodiments, the polymerase can include a hot-start polymerase or an aptamer-based polymerase that optionally can be reactivated.

The terms “target primer” or “target-specific primer” and variations thereof refer to primers that are complementary to a binding site sequence. Target primers are generally a single stranded or double-stranded polynucleotide, typically an oligonucleotide, that includes at least one sequence that is at least partially complementary to a target nucleic acid sequence.

“Forward primer binding site” and “reverse primer binding site” refers to the regions on the template DNA and/or the amplicon to which the forward and reverse primers bind. The primers act to delimit the region of the original template polynucleotide which is exponentially amplified during amplification. In some embodiments, additional primers may bind to the region 5′ of the forward primer and/or reverse primers. Where such additional primers are used, the forward primer binding site and/or the reverse primer binding site may encompass the binding regions of these additional primers as well as the binding regions of the primers themselves. For example, in some embodiments, the method may use one or more additional primers which bind to a region that lies 5′ of the forward and/or reverse primer binding region. Such a method was disclosed, for example, in WO0028082 which discloses the use of “displacement primers” or “outer primers”.

A ‘barcode’ nucleic acid identification sequence can be incorporated into a nucleic acid primer or linked to a primer to enable independent sequencing and identification to be associated with one another via a barcode which relates information and identification that originated from molecules that existed within the same sample. There are numerous techniques that can be used to attach barcodes to the nucleic acids within a discrete entity. For example, the target nucleic acids may or may not be first amplified and fragmented into shorter pieces. The molecules can be combined with discrete entities, e.g., droplets, containing the barcodes. The barcodes can then be attached to the molecules using, for example, splicing by overlap extension. In this approach, the initial target molecules can have “adaptor” sequences added, which are molecules of a known sequence to which primers can be synthesized. When combined with the barcodes, primers can be used that are complementary to the adaptor sequences and the barcode sequences, such that the product amplicons of both target nucleic acids and barcodes can anneal to one another and, via an extension reaction such as DNA polymerization, be extended onto one another, generating a double-stranded product including the target nucleic acids attached to the barcode sequence. Alternatively, the primers that amplify that target can themselves be barcoded so that, upon annealing and extending onto the target, the amplicon produced has the barcode sequence incorporated into it. This can be applied with a number of amplification strategies, including specific amplification with PCR or non-specific amplification with, for example, MDA. An alternative enzymatic reaction that can be used to attach barcodes to nucleic acids is ligation, including blunt or sticky end ligation. In this approach, the DNA barcodes are incubated with the nucleic acid targets and ligase enzyme, resulting in the ligation of the barcode to the targets. The ends of the nucleic acids can be modified as needed for ligation by a number of techniques, including by using adaptors introduced with ligase or fragments to enable greater control over the number of barcodes added to the end of the molecule.

A barcode sequence can additionally be incorporated into microfluidic beads to decorate the bead with identical sequence tags. Such tagged beads can be inserted into microfluidic droplets and via droplet PCR amplification, tag each target amplicon with the unique bead barcode. Such barcodes can be used to identify specific droplets upon a population of amplicons originated from. This scheme can be utilized when combining a microfluidic droplet containing single individual cell with another microfluidic droplet containing a tagged bead. Upon collection and combination of many microfluidic droplets, amplicon sequencing results allow for assignment of each product to unique microfluidic droplets. In a typical implementation, we use barcodes on the Mission Bio Tapestri™ beads to tag and then later identify each droplet's amplicon content. The use of barcodes is described in U.S. patent application Ser. No. 15/940,850 filed Mar. 29, 2018 by Abate, A. et al., entitled ‘Sequencing of Nucleic Acids via Barcoding in Discrete Entities’, incorporated by reference herein.

In some embodiments, it may be advantageous to introduce barcodes into discrete entities, e.g., microdroplets, on the surface of a bead, such as a solid polymer bead or a hydrogel bead. These beads can be synthesized using a variety of techniques. For example, using a mix-split technique, beads with many copies of the same, random barcode sequence can be synthesized. This can be accomplished by, for example, creating a plurality of beads including sites on which DNA can be synthesized. The beads can be divided into four collections and each mixed with a buffer that will add a base to it, such as an A, T, G, or C. By dividing the population into four subpopulations, each subpopulation can have one of the bases added to its surface. This reaction can be accomplished in such a way that only a single base is added and no further bases are added. The beads from all four subpopulations can be combined and mixed together, and divided into four populations a second time. In this division step, the beads from the previous four populations may be mixed together randomly. They can then be added to the four different solutions, adding another, random base on the surface of each bead. This process can be repeated to generate sequences on the surface of the bead of a length approximately equal to the number of times that the population is split and mixed. If this was done 10 times, for example, the result would be a population of beads in which each bead has many copies of the same random 10-base sequence synthesized on its surface. The sequence on each bead would be determined by the particular sequence of reactors it ended up in through each mix-spit cycle.

A barcode may further comprise a ‘unique identification sequence’ (UMI). A UMI is a nucleic acid having a sequence which can be used to identify and/or distinguish one or more first molecules to which the UMI is conjugated from one or more second molecules. UMIs are typically short, e.g., about 5 to 20 bases in length, and may be conjugated to one or more target molecules of interest or amplification products thereof. UMIs may be single or double stranded. In some embodiments, both a nucleic acid barcode sequence and a UMI are incorporated into a nucleic acid target molecule or an amplification product thereof. Generally, a UMI is used to distinguish between molecules of a similar type within a population or group, whereas a nucleic acid barcode sequence is used to distinguish between populations or groups of molecules. In some embodiments, where both a UMI and a nucleic acid barcode sequence are utilized, the UMI is shorter in sequence length than the nucleic acid barcode sequence.

The terms “identity” and “identical” and their variants, as used herein, when used in reference to two or more nucleic acid sequences, refer to similarity in sequence of the two or more sequences (e.g., nucleotide or polypeptide sequences). In the context of two or more homologous sequences, the percent identity or homology of the sequences or subsequences thereof indicates the percentage of all monomeric units (e.g., nucleotides or amino acids) that are the same (i.e., about 70% identity, preferably 75%, 80%, 85%, 90%, 95%, 97%, 98% or 99% identity). The percent identity can be over a specified region, when compared and aligned for maximum correspondence over a comparison window, or designated region as measured using a BLAST or BLAST 2.0 sequence comparison algorithms with default parameters described below, or by manual alignment and visual inspection. Sequences are said to be “substantially identical” when there is at least 85% identity at the amino acid level or at the nucleotide level. Preferably, the identity exists over a region that is at least about 25, 50, or 100 residues in length, or across the entire length of at least one compared sequence. A typical algorithm for determining percent sequence identity and sequence similarity are the BLAST and BLAST 2.0 algorithms, which are described in Altschul et al, Nuc. Acids Res. 25:3389-3402 (1977). Other methods include the algorithms of Smith & Waterman, Adv. Appl. Math. 2:482 (1981), and Needleman & Wunsch, J. Mol. Biol. 48:443 (1970), etc. Another indication that two nucleic acid sequences are substantially identical is that the two molecules or their complements hybridize to each other under stringent hybridization conditions.

The terms “nucleic acid,” “polynucleotides,” and “oligonucleotides” refers to biopolymers of nucleotides and, unless the context indicates otherwise, includes modified and unmodified nucleotides, and both DNA and RNA, and modified nucleic acid backbones. For example, in certain embodiments, the nucleic acid is a peptide nucleic acid (PNA) or a locked nucleic acid (LNA). Typically, the methods as described herein are performed using DNA as the nucleic acid template for amplification. However, nucleic acid whose nucleotide is replaced by an artificial derivative or modified nucleic acid from natural DNA or RNA is also included in the nucleic acid of the present invention insofar as it functions as a template for synthesis of complementary chain. The nucleic acid of the present invention is generally contained in a biological sample. The biological sample includes animal, plant or microbial tissues, cells, cultures and excretions, or extracts therefrom. In certain aspects, the biological sample includes intracellular parasitic genomic DNA or RNA such as virus or mycoplasma. The nucleic acid may be derived from nucleic acid contained in said biological sample. For example, genomic DNA, or cDNA synthesized from mRNA, or nucleic acid amplified on the basis of nucleic acid derived from the biological sample, are preferably used in the described methods. Unless denoted otherwise, whenever a oligonucleotide sequence is represented, it will be understood that the nucleotides are in 5′ to 3′ order from left to right and that “A” denotes deoxyadenosine, “C” denotes deoxycytidine, “G” denotes deoxyguanosine, “T” denotes thymidine, and “U’ denotes deoxyuridine. Oligonucleotides are said to have “5′ ends” and “3′ ends” because mononucleotides are typically reacted to form oligonucleotides via attachment of the 5′ phosphate or equivalent group of one nucleotide to the 3′ hydroxyl or equivalent group of its neighboring nucleotide, optionally via a phosphodiester or other suitable linkage.

A template nucleic acid is a nucleic acid serving as a template for synthesizing a complementary chain in a nucleic acid amplification technique. A complementary chain having a nucleotide sequence complementary to the template has a meaning as a chain corresponding to the template, but the relationship between the two is merely relative. That is, according to the methods described herein a chain synthesized as the complementary chain can function again as a template. That is, the complementary chain can become a template. In certain embodiments, the template is derived from a biological sample, e.g., plant, animal, virus, micro-organism, bacteria, fungus, etc. In certain embodiments, the animal is a mammal, e.g., a human patient. A template nucleic acid typically comprises one or more target nucleic acid. A target nucleic acid in exemplary embodiments may comprise any single or double-stranded nucleic acid sequence that can be amplified or synthesized according to the disclosure, including any nucleic acid sequence suspected or expected to be present in a sample.

Primers and oligonucleotides used in embodiments herein comprise nucleotides. A nucleotide comprises any compound, including without limitation any naturally occurring nucleotide or analog thereof, which can bind selectively to, or can be polymerized by, a polymerase. Typically, but not necessarily, selective binding of the nucleotide to the polymerase is followed by polymerization of the nucleotide into a nucleic acid strand by the polymerase; occasionally however the nucleotide may dissociate from the polymerase without becoming incorporated into the nucleic acid strand, an event referred to herein as a “non-productive” event. Such nucleotides include not only naturally occurring nucleotides but also any analogs, regardless of their structure, that can bind selectively to, or can be polymerized by, a polymerase. While naturally occurring nucleotides typically comprise base, sugar and phosphate moieties, the nucleotides of the present disclosure can include compounds lacking any one, some or all of such moieties. For example, the nucleotide can optionally include a chain of phosphorus atoms comprising three, four, five, six, seven, eight, nine, ten or more phosphorus atoms. In some embodiments, the phosphorus chain can be attached to any carbon of a sugar ring, such as the 5′ carbon. The phosphorus chain can be linked to the sugar with an intervening O or S. In one embodiment, one or more phosphorus atoms in the chain can be part of a phosphate group having P and O. In another embodiment, the phosphorus atoms in the chain can be linked together with intervening O, NH, S, methylene, substituted methylene, ethylene, substituted ethylene, CNH₂, C(O), C(CH₂), CH₂CH₂, or C(OH)CH₂R (where R can be a 4-pyridine or 1-imidazole). In one embodiment, the phosphorus atoms in the chain can have side groups having O, BH₃, or S. In the phosphorus chain, a phosphorus atom with a side group other than O can be a substituted phosphate group. In the phosphorus chain, phosphorus atoms with an intervening atom other than O can be a substituted phosphate group. Some examples of nucleotide analogs are described in Xu, U.S. Pat. No. 7,405,281.

In some embodiments, the nucleotide comprises a label and referred to herein as a “labeled nucleotide”; the label of the labeled nucleotide is referred to herein as a “nucleotide label”. In some embodiments, the label can be in the form of a fluorescent moiety (e.g. dye), luminescent moiety, or the like attached to the terminal phosphate group, i.e., the phosphate group most distal from the sugar. Some examples of nucleotides that can be used in the disclosed methods and compositions include, but are not limited to, ribonucleotides, deoxyribonucleotides, modified ribonucleotides, modified deoxyribonucleotides, ribonucleotide polyphosphates, deoxyribonucleotide polyphosphates, modified ribonucleotide polyphosphates, modified deoxyribonucleotide polyphosphates, peptide nucleotides, modified peptide nucleotides, metallonucleosides, phosphonate nucleosides, and modified phosphate-sugar backbone nucleotides, analogs, derivatives, or variants of the foregoing compounds, and the like. In some embodiments, the nucleotide can comprise non-oxygen moieties such as, for example, thio- or borano-moieties, in place of the oxygen moiety bridging the alpha phosphate and the sugar of the nucleotide, or the alpha and beta phosphates of the nucleotide, or the beta and gamma phosphates of the nucleotide, or between any other two phosphates of the nucleotide, or any combination thereof. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group can include sulfur substitutions for the various oxygens, e.g. α-thio-nucleotide 5′-triphosphates. For a review of nucleic acid chemistry, see: Shabarova, Z. and Bogdanov, A. Advanced Organic Chemistry of Nucleic Acids, VCH, New York, 1994.

Any nucleic acid amplification method may be utilized, such as a PCR-based assay, e.g., quantitative PCR (qPCR), or an isothermal amplification may be used to detect the presence of certain nucleic acids, e.g., genes, of interest, present in discrete entities or one or more components thereof, e.g., cells encapsulated therein. Such assays can be applied to discrete entities within a microfluidic device or a portion thereof or any other suitable location. The conditions of such amplification or PCR-based assays may include detecting nucleic acid amplification over time and may vary in one or more ways.

The number of amplification/PCR primers that may be added to a microdroplet may vary. The number of amplification or PCR primers that may be added to a microdroplet may range from about 1 to about 500 or more, e.g., about 2 to 100 primers, about 2 to 10 primers, about 10 to 20 primers, about 20 to 30 primers, about 30 to 40 primers, about 40 to 50 primers, about 50 to 60 primers, about 60 to 70 primers, about 70 to 80 primers, about 80 to 90 primers, about 90 to 100 primers, about 100 to 150 primers, about 150 to 200 primers, about 200 to 250 primers, about 250 to 300 primers, about 300 to 350 primers, about 350 to 400 primers, about 400 to 450 primers, about 450 to 500 primers, or about 500 primers or more.

One or both primers of a primer set may comprise a barcode sequence. In some embodiments, one or both primers comprise a barcode sequence and a unique molecular identifier (UMI). In some embodiments, where both a UMI and a nucleic acid barcode sequence are utilized, the UMI is incorporated into the target nucleic acid or an amplification product thereof prior to the incorporation of the nucleic acid barcode sequence. In some embodiments, where both a UMI and a nucleic acid barcode sequence are utilized, the nucleic acid barcode sequence is incorporated into the UMI or an amplification product thereof subsequent to the incorporation of the UMI into a target nucleic acid or an amplification product thereof.

One or both primer of a primer set may also be attached or conjugated to an affinity reagent. In some embodiments, individual cells, for example, are isolated in discrete entities, e.g., droplets. These cells may be lysed and their nucleic acids barcoded. This process can be performed on a large number of single cells in discrete entities with unique barcode sequences enabling subsequent deconvolution of mixed sequence reads by barcode to obtain single cell information. This approach provides a way to group together nucleic acids originating from large numbers of single cells. Additionally, affinity reagents such as antibodies can be conjugated with nucleic acid labels, e.g., oligonucleotides including barcodes, which can be used to identify antibody type, e.g., the target specificity of an antibody. These reagents can then be used to bind to the proteins within or on cells, thereby associating the nucleic acids carried by the affinity reagents to the cells to which they are bound. These cells can then be processed through a barcoding workflow as described herein to attach barcodes to the nucleic acid labels on the affinity reagents. Techniques of library preparation, sequencing, and bioinformatics may then be used to group the sequences according to cell/discrete entity barcodes. Any suitable affinity reagent that can bind to or recognize a biological sample or portion or component thereof, such as a protein, a molecule, or complexes thereof, may be utilized in connection with these methods. The affinity reagents may be labeled with nucleic acid sequences that relates their identity, e.g., the target specificity of the antibodies, permitting their detection and quantitation using the barcoding and sequencing methods described herein. Exemplary affinity reagents can include, for example, antibodies, antibody fragments, Fabs, scFvs, peptides, drugs, etc. or combinations thereof. The affinity reagents, e.g., antibodies, can be expressed by one or more organisms or provided using a biological synthesis technique, such as phage, mRNA, or ribosome display. The affinity reagents may also be generated via chemical or biochemical means, such as by chemical linkage using N-Hydroxysuccinimide (NETS), click chemistry, or streptavidin-biotin interaction, for example. The oligo-affinity reagent conjugates can also be generated by attaching oligos to affinity reagents and hybridizing, ligating, and/or extending via polymerase, etc., additional oligos to the previously conjugated oligos. An advantage of affinity reagent labeling with nucleic acids is that it permits highly multiplexed analysis of biological samples. For example, large mixtures of antibodies or binding reagents recognizing a variety of targets in a sample can be mixed together, each labeled with its own nucleic acid sequence. This cocktail can then be reacted to the sample and subjected to a barcoding workflow as described herein to recover information about which reagents bound, their quantity, and how this varies among the different entities in the sample, such as among single cells. The above approach can be applied to a variety of molecular targets, including samples including one or more of cells, peptides, proteins, macromolecules, macromolecular complexes, etc. The sample can be subjected to conventional processing for analysis, such as fixation and permeabilization, aiding binding of the affinity reagents. To obtain highly accurate quantitation, the unique molecular identifier (UMI) techniques described herein can also be used so that affinity reagent molecules are counted accurately. This can be accomplished in a number of ways, including by synthesizing UMIs onto the labels attached to each affinity reagent before, during, or after conjugation, or by attaching the UMIs microfluidically when the reagents are used. Similar methods of generating the barcodes, for example, using combinatorial barcode techniques as applied to single cell sequencing and described herein, are applicable to the affinity reagent technique. These techniques enable the analysis of proteins and/or epitopes in a variety of biological samples to perform, for example, mapping of epitopes or post translational modifications in proteins and other entities or performing single cell proteomics. For example, using the methods described herein, it is possible to generate a library of labeled affinity reagents that detect an epitope in all proteins in the proteome of an organism, label those epitopes with the reagents, and apply the barcoding and sequencing techniques described herein to detect and accurately quantitate the labels associated with these epitopes.

Primers may contain primers for one or more nucleic acid of interest, e.g. one or more genes of interest. The number of primers for genes of interest that are added may be from about one to 500, e.g., about 1 to 10 primers, about 10 to 20 primers, about 20 to 30 primers, about 30 to 40 primers, about 40 to 50 primers, about 50 to 60 primers, about 60 to 70 primers, about 70 to 80 primers, about 80 to 90 primers, about 90 to 100 primers, about 100 to 150 primers, about 150 to 200 primers, about 200 to 250 primers, about 250 to 300 primers, about 300 to 350 primers, about 350 to 400 primers, about 400 to 450 primers, about 450 to 500 primers, or about 500 primers or more. Primers and/or reagents may be added to a discrete entity, e.g., a microdroplet, in one step, or in more than one step. For instance, the primers may be added in two or more steps, three or more steps, four or more steps, or five or more steps. Regardless of whether the primers are added in one step or in more than one step, they may be added after the addition of a lysing agent, prior to the addition of a lysing agent, or concomitantly with the addition of a lysing agent. When added before or after the addition of a lysing agent, the PCR primers may be added in a separate step from the addition of a lysing agent. In some embodiments, the discrete entity, e.g., a microdroplet, may be subjected to a dilution step and/or enzyme inactivation step prior to the addition of the PCR reagents. Exemplary embodiments of such methods are described in PCT Publication No. WO 2014/028378, the disclosure of which is incorporated by reference herein in its entirety and for all purposes.

A primer set for the amplification of a target nucleic acid typically includes a forward primer and a reverse primer that are complementary to a target nucleic acid or the complement thereof. In some embodiments, amplification can be performed using multiple target-specific primer pairs in a single amplification reaction, wherein each primer pair includes a forward target-specific primer and a reverse target-specific primer, where each includes at least one sequence that substantially complementary or substantially identical to a corresponding target sequence in the sample, and each primer pair having a different corresponding target sequence. Accordingly, certain methods herein are used to detect or identify multiple target sequences from a single cell sample.

An exemplary embodiment is a system and method for detection of a target nucleic acid from a single cell, the method including, independent of order presented, the following steps: selecting one or more target nucleic acid sequence of interest in an individual cell, where the target nucleic acid sequence is complementary to a nucleic acid in a cell; providing a sample having on or more individual single cells; encapsulating one or more individual cell in a reaction mixture comprising a protease; incubating the encapsulated cell with the protease in the drop to produce a cell lysate; providing one or more nucleic acid amplification primer sets, wherein each primer set is complementary to a target nucleic acid and at least one primer of a nucleic acid amplification primer set comprises a barcode sequence; providing one or more universal bases in an nucleic acid amplification reaction mixture; performing a nucleic acid amplification reaction using the reaction mixture comprising the universal bases to form an amplification product from the nucleic acid of a single cell, where the amplification product has amplicons of one or more target nucleic acid sequence; and optionally the following, providing an affinity reagent that comprises a nucleic acid sequence complementary to the barcode sequence of one of more nucleic acid primer of a primer set, where the affinity reagent comprising said nucleic acid sequence complementary to the barcode sequence is capable of binding to a nucleic acid amplification primer set comprising a barcode sequence; contacting an affinity reagent to the amplification product comprising amplicons of one or more target nucleic acid sequence under conditions sufficient for binding of the affinity reagent to the target nucleic acid to form an affinity reagent bound target nucleic acid; and determining the identity of the target nucleic acids by sequencing the first bar code and second bar code.

A fundamental challenge in precision medicine has been improving the understanding of cancer heterogeneity and clonal evolution, which has major implications in targeted therapy selection and disease monitoring. However, current bulk sequencing methods are unable to unambiguously identify rare pathogenic or drug-resistant cell populations and determine whether mutations co-occur within the same cell. Single-cell sequencing has the potential to provide unique insights on the cellular and genetic composition, drivers, and signatures of cancer at unparalleled sensitivity. Previously we have developed a high-throughput single-cell DNA analysis platform (Tapestri™, Mission Bio, South San Francisco Calif.) that leverages droplet microfluidics and a multiplex-PCR based targeted DNA sequencing approach, and demonstrated the generation of high-resolution maps of clonal architecture from acute myeloid leukemia (AML) tumors.

An exemplary embodiment is a system and method for detection of a target nucleic acid from a single cell, the method including, independent of order presented, the following steps: selecting one or more target nucleic acid sequence, where optionally, the target nucleic acid sequence is complementary to a nucleic acid in a cell of interest; providing a sample having on or more individual single cells; encapsulating one or more individual cell in a reaction mixture comprising a protease; incubating the encapsulated cell with the protease in the drop to produce a cell lysate; providing one or more nucleic acid amplification primer sets, wherein each primer set is complementary to a target nucleic acid and at least one primer of a nucleic acid amplification primer set comprises a barcode sequence; performing a nucleic acid amplification reaction to form an amplification product from the nucleic acid of a single cell, where the amplification product has amplicons of one or more target nucleic acid sequence; providing an affinity reagent that comprises a nucleic acid sequence complementary to the barcode sequence of one of more nucleic acid primer of a primer set, where the affinity reagent comprising said nucleic acid sequence complementary to the barcode sequence is capable of binding to a nucleic acid amplification primer set comprising a barcode sequence; contacting an affinity reagent to the amplification product comprising amplicons of one or more target nucleic acid sequence under conditions sufficient for binding of the affinity reagent to the target nucleic acid to form an affinity reagent bound target nucleic acid; and characterizing a mutation or translocation associated with the target nucleic acid by nucleic acid sequencing.

In another aspect, certain affinity reagent barcoding techniques described herein can be used to detect and quantitate protein-protein interactions. For example, proteins that interact can be labeled with nucleic acid sequences and reacted with one another. If the proteins interact by, for example, binding one another, their associated labels are localized to the bound complex, whereas proteins that do not interact will remain unbound from one another. The sample can then be isolated in discrete entities, such as microfluidic droplets, and subjected to fusion amplification/PCR or barcoding of the nucleic acid labels. In the case that proteins interact, a given barcode group will contain nucleic acids including the labels of both interacting proteins, since those nucleic acids would have ended up in the same compartment and been barcoded by the same barcode sequence. In contrast, proteins that do not interact will statistically end up in different compartments and, thus, will not cluster into the same barcode group post sequencing. This allows identification of which proteins interact by clustering the data according to barcode and detecting all affinity reagent labels in the group. A purification step can also be implemented to remove unbound affinity reagents prior to isolation in discrete entities, which discards sequences that yield no interaction data. Alternatively, using the fusion approach, such as pairwise fusions post-encapsulation, amplification can be used to selectively amplify fused products, effectively diluting away unfused molecules and enriching for fusions, making the sequencing more efficient for detecting interacting proteins.

Accordingly, certain embodiments the invention provide methods for linking and amplifying nucleic acids conjugated to proteins (e.g antibodies). An exemplary method includes: (a) incubating a population of nucleic acid barcode sequence-conjugated proteins under conditions sufficient for a plurality of the proteins to interact, bringing the nucleic acid barcode sequences on the interacting proteins in proximity to each other; (b) encapsulating the population of nucleic acid barcode sequence-conjugated proteins in a plurality of discrete entities such that interacting proteins are co-encapsulated, if present; (c) using a microfluidic device to combine in a discrete entity contents of one of the plurality of first discrete entities and reagents sufficient for amplification and linkage of the nucleic acid barcode sequences on the interacting proteins, if present; and (d) subjecting the discrete entity to conditions sufficient for the amplification and linkage of the nucleic acid barcode sequences on the interacting proteins, if present.

### Proteomics

Another objective of some embodiments herein is to provide a sensitive, accurate, and comprehensive characterization of proteins in large numbers of single cells.

Certain methods provided herein utilize specific antibodies to detect epitopes of interest. In some embodiments, antibodies are labeled with sequence tags that can be read out with microfluidic barcoding and DNA sequencing. This and related implementations are used herein to characterize cell surface proteins of different cell types at the single-cell level.

In some embodiments, a barcode identity is encoded by its full nucleobase sequence and thus confers a combinatorial tag space far exceeding what is possible with conventional approaches using fluorescence. A modest tag length of ten bases provides over a million unique sequences, sufficient to label an antibody against every epitope in the human proteome. Indeed, with this approach, the limit to multiplexing is not the availability of unique tag sequences but, rather, that of specific antibodies that can detect the epitopes of interest in a multiplexed reaction.

In some implementations, cells are bound with antibodies against the different target epitopes, as in conventional immunostaining, except that the antibodies are labeled with barcodes.

In practice, when an antibody binds its target the DNA barcode tag is carried with it and thus allows the presence of the target to be inferred based on the presence of the barcode. In some implementations, counting barcode tags provides an estimate of the different epitopes present in the cell.

Other embodiments implementations are used to distinguish particular cells by their protein expression profiles. Some embodiments of DNA-tagged antibodies provided herein have multiple advantages for profiling proteins in single cells.

A primary advantage of these implementations is the ability to amplify low-abundance tags to make them detectable with sequencing. Another advantage in some implementations is the capability of using molecular indices for quantitative results. Some implementations also have essentially limitless multiplexing capabilities.

Some embodiments utilize solid beads having an alternate chemistry where the primers to be used are in solution and contain a PCR annealing sequence embedded, or ‘handle’, that allows hybridization to primers. In some implementations, the handle is a specific tail 5′ upstream of the target sequence and this handle is complimentary to bead barcoded oligo and serves as a PCR extension bridge to link the target amplicon to the bead barcode library primer sequence. The solid beads may contain primers that can anneal to the PCR handle on the primers.

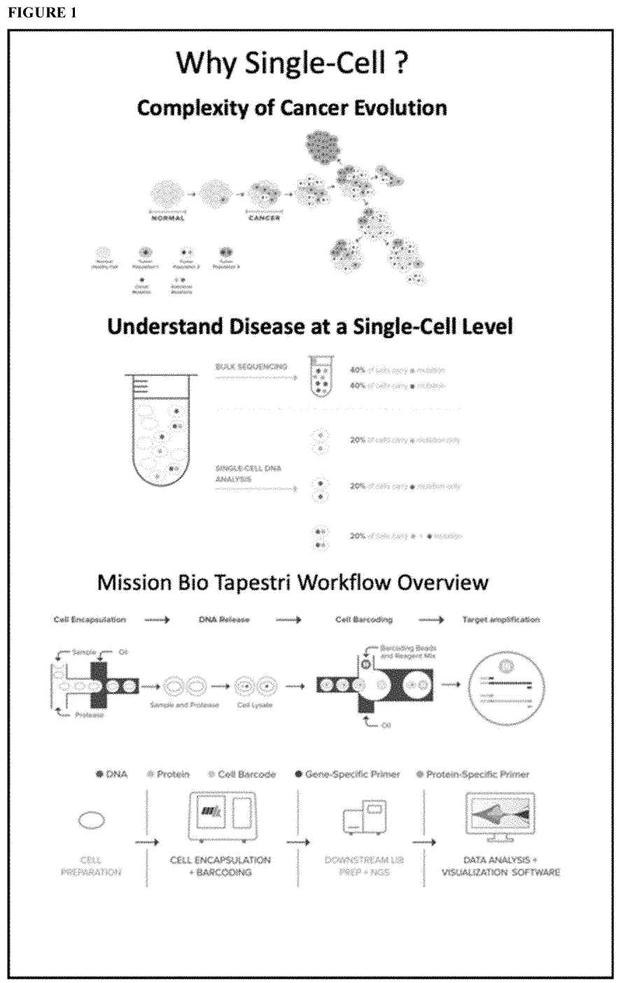

One embodiment is for a method for adding a barcode identification sequence linked to an antibody, the method comprising the steps: i) an initial hybridization of a target gDNA to a) a forward primer comprising a first read sequence adjacent to a first cell barcode, adjacent to a constant region 1, adjacent to a second cell bar code, which is adjacent to a constant region 2 and b) a reverse primer comprising a sequence complementary to the target genomic DNA, which is adjacent to unique molecular identifier, which is adjacent to an antibody tag sequence, which is adjacent to a second unique molecular identifier; which is adjacent to a second read sequence, and performing a barcoding PCR reaction. The resulting amplicon comprises a first read sequence adjacent to a first cell barcode, adjacent to a constant region 1, adjacent to a second cell bar code, which is adjacent to a constant region 2, which is adjacent to the forward primer sequence, which is adjacent to an insert sequence of length ‘n’, which is adjacent to a reverse primer comprising a sequence complementary to the target genomic DNA, which is adjacent to unique molecular identifier, which is adjacent to an antibody tag sequence, which is adjacent to a second unique molecular identifier; which is adjacent to a second read sequence. An additional library creation PCR step is typically used in some embodiments to further attach indexing and identification sequences (see for example FIG. 1).

Antibody libraries can be created from antibody stained cells, and these can identify and characterized by sequencing.

In another aspect, some implementations provided herein can be used to detect and characterize the mRNA and protein expression pattern in single cell.

In some implementations, certain affinity reagent barcoding techniques described herein can be used to detect and quantitate protein-protein interactions. For example, proteins that interact can be labeled with nucleic acid sequences and reacted with one another. If the proteins interact by, for example, binding one another, their associated labels are localized to the bound complex, whereas proteins that do not interact will remain unbound from one another.

The sample can then be isolated in discrete entities, such as microfluidic droplets, and subjected to fusion amplification/PCR or barcoding of the nucleic acid labels. In the case that proteins interact, a given barcode group will contain nucleic acids including the labels of both interacting proteins, since those nucleic acids would have ended up in the same compartment and been barcoded by the same barcode sequence. In contrast, proteins that do not interact will statistically end up in different compartments and, thus, will not cluster into the same barcode group post sequencing. This allows identification of which proteins interact by clustering the data according to barcode and detecting all affinity reagent labels in the group. A purification step can also be implemented to remove unbound affinity reagents prior to isolation in discrete entities, which discards sequences that yield no interaction data. Alternatively, using the fusion approach, such as pairwise fusions post-encapsulation, amplification can be used to selectively amplify fused products, effectively diluting away unfused molecules and enriching for fusions, making the sequencing more efficient for detecting interacting proteins.

Certain embodiments the invention provide methods for linking and amplifying nucleic acids conjugated to proteins, such as antibodies, enzymes, receptors, and the like. An exemplary method includes: (a) incubating a population of nucleic acid barcode sequence-conjugated proteins under conditions sufficient for a plurality of the proteins to interact, bringing the nucleic acid barcode sequences on the interacting proteins in proximity to each other; (b) encapsulating the population of nucleic acid barcode sequence-conjugated proteins in a plurality of discrete entities such that interacting proteins are co-encapsulated, if present; (c) using a microfluidic device to combine in a discrete entity contents of one of the plurality of first discrete entities and reagents sufficient for amplification and linkage of the nucleic acid barcode sequences on the interacting proteins, if present; and (d) subjecting the discrete entity to conditions sufficient for the amplification and linkage of the nucleic acid barcode sequences on the interacting proteins, if present.

Some embodiments utilize solid beads having an alternate chemistry where the primers to be used are in solution and contain a PCR annealing sequence embedded, or ‘handle’, that allows hybridization to primers. In some implementations, the handle is a specific tail 5′ upstream of the target sequence and this handle is complimentary to bead barcoded oligo and serves as a PCR extension bridge to link the target amplicon to the bead barcode library primer sequence. The solid beads may contain primers that can anneal to the PCR handle on the primers.

Other aspects of the invention may be described in the follow embodiments:

1. An apparatus or system for performing a method described herein.

2. A composition or reaction mixture for performing a method described herein.

3. A transcriptome library generated according to a method described herein.

5. A genomic and transcriptome library generated according to a method described herein.

6. An antibody library described herein.

7. A kit for performing a method described herein.

8. A cell population selected by the methods described herein.

9. A method for preparing an antibody library and a DNA library which can be paired based on the cell barcode.

10. A method for preparing an antibody library and a RNA library which can be paired based on the cell barcode.

11. A method for preparing an antibody library, DNA library, and RNA library which can be paired based on the cell barcode.

12. A method according to one or more Figure or Description provided herein.

The following Examples are included for illustration and not limitation.

### Example I

### Methods to Identify Tumor Heterogeneity

In this Example we present subclone identification method using data generated on the Tapestri™ single-cell DNA platform and analyzed by Tapestri™ analytical workflow. The pipeline steps involve obtaining raw reads from the sequencer, removing adapters, aligning and mapping the reads, calling individual cells, and identifying genetic variants within each cell. After filtering for high quality variants, we then filter for data completeness to ensure only high quality data is used in downstream processing. The variant-cell matrix is then subjected to identification of subclones. Top variants defined the signature of each subclone are also identified. To validate our methodology, we used two different targeted sequencing panels on model systems with known truth mutations. Our pipeline shows the distinct clusters correlating with titration and cell line ratios. Cluster associated signature mutations were also identified. The pipeline can be used for multi sample analysis with timeseries data from diagnosis to relapse or from primary site to metastasis to understand clonal diversity. These data demonstrate the utility of the Tapestri™ platform, the analytical pipeline, and associated data visualization capability. Our approach addresses key issues of identifying rare subpopulations of cells down to 0. 1 0 0, and transforms the ability to accurately characterize clonal heterogeneity in tumor samples. This high throughput method advances research efforts to improve patient stratification and therapy selection for various cancer indications. To understand which mutations are real drivers of AML versus one that are just passengers or contributors, longitudinal analysis using AML panel was performed to reveal the therapy resistant clones. Pre-treatment leukemia sample, on-treatment and relapse BM samples were analyzed by single-cell sequencing. The analysis resolves evolution of 3 subclones basing on combination of 4 mutations, helps to understand clonal composition of cancer for making dynamic changes in treatment. With the analytical method demonstrated above, we show the distinct clusters correlating with titration and cell line ratio. We were also able to identify the cluster associated signature mutations. These data demonstrate the utility of the Tapestri™ platform, and the analytical pipeline, and associated data visualization capability. Our approach has the potential to address the key issues of identifying rare subpopulations of cells and transforms our ability to accurately characterize clonal heterogeneity in tumor samples. This high throughput method of accurately characterizing clonal populations should lead to improved patient stratification and therapy selection for various cancer indications.

The data described in the previous embodiment highlight the capability of identifying the biologically relevant variants at the moment of the diagnosis, but also how the treatment positively select resistant cellular clones based on the mutation signature. This positions the Tapestri™ platform as the only tool available to study how genetic variants co-exist and which combinations are sensitive and resistant to certain treatments. Thus, it helps in the diagnostic precision, treatment follow up and new target identification and drug development.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A method of identifying and characterizing clonal sub populations of cells, the method comprising the steps of:
a) conjugating barcode sequences flanked by PCR priming sites onto antibodies, wherein a barcode sequence is specific to an antibody;
b) performing a cell staining step using the barcode-conjugated antibodies;
c) partitioning or separating individual cells or nuclei, and encapsulating one or more individual cell(s) or nuclei, in a reaction mixture comprising a protease and/or reverse transcriptase;
d) incubating the encapsulated cell with the protease in the drop to produce cDNA in a cell lysate with released chromatin;
e) providing one or more nucleic acid amplification primer sets, wherein one or more primer of a primer set comprises a barcode identification sequence associated with an antibody;
f) performing a nucleic acid amplification reaction to produce one or more amplicons;
g) providing an affinity reagent that comprises a nucleic acid sequence complementary to the identification barcode sequence of one of more nucleic acid primer of a primer set, wherein said affinity reagent comprising said nucleic acid sequence complementary to the identification barcode sequence is capable of binding to a nucleic acid amplification primer set comprising a barcode identification sequence;
h) contacting an affinity reagent to the amplification product comprising amplicons of one or more target nucleic acid sequence under conditions sufficient for binding of the affinity reagent to the target nucleic acid to form an affinity reagent bound target nucleic acid; and
i) determining the identity and characterizing one or more protein by sequencing a barcode of an amplicon.

2. A method of claim 1, wherein signature mutations are identified at a single-cell level

3. A method for detection of gene expression in a nucleic acid sample from a single cell, the method comprising:
a. selecting one or more target nucleic acid sequence in an individual cell, where the target nucleic acid sequence is contained in a DNA or RNA; providing a sample having one or more individual single cell;
b. encapsulating an individual cell in a drop;
c. incubating the encapsulated cell in presence of protease and/or reverse transcriptase in the drop to produce cDNA and a cell lysate;
d. providing a nucleic acid amplification primer set complementary to a target nucleic acid, where at least one primer of the nucleic acid amplification primer set comprises a barcode identification sequence;
e. performing a reverse transcription and nucleic acid amplification reaction to form an amplification product from the nucleic acid of a single cell; and
f. determining whether the target nucleic acid is expressed if the target nucleic acid comprises a transcript.

4. A method according to claim 1, further comprising:
a) providing an affinity reagent that comprises a nucleic acid sequence complementary to a barcode sequence of one of more nucleic acid primer, where the affinity reagent comprising said nucleic acid sequence complementary to the barcode sequence is capable of binding to a nucleic acid amplification primer comprising a barcode sequence; and
b) contacting an affinity reagent to the amplification product comprising amplicons under conditions sufficient for binding of the affinity reagent to the target nucleic acid to form an affinity reagent bound target nucleic acid.

5. A method according to claim 1, further comprising nucleic acid sequencing of an amplification product or amplicon to determine whether the target nucleic acid is present.

6. A method according to claim 1, that includes a reverse transcriptase and comprises performing reverse transcription to produce a reverse transcription product.

7. A method according to claim 1, that includes a reverse transcriptase and comprises performing reverse transcription to produce a reverse transcription product before a nucleic acid amplification step.

8. A method according to claim 1, that includes a reverse transcriptase and comprises performing reverse transcription on the RNA to produce a reverse transcription product and amplifying the reverse transcription product, wherein performing reverse transcription and amplifying occur in a single step.

9. A method according to claim 1, further comprising performing a nucleic acid sequencing reaction of an amplification product.

10. A method according to claim 1, wherein the affinity reagent comprises a bead or the like.

11. A method according to claim 1, comprising determining and characterizing the expression of one or more cell surface protein.

12. A method according to claim 1 further comprising preparing an antibody library and a DNA library which can be paired based on the cell barcode.

13. A method according to claim 1 further comprising preparing an antibody library and a RNA library which can be paired based on the cell barcode.

14. A method according to claim 1 further comprising preparing an antibody library, DNA library, and RNA library which can be paired based on the cell barcode.

15. A method according to claim 1 wherein the affinity reagent comprises a bead, solid support, or the like.

16. A method according to FIG. 1.

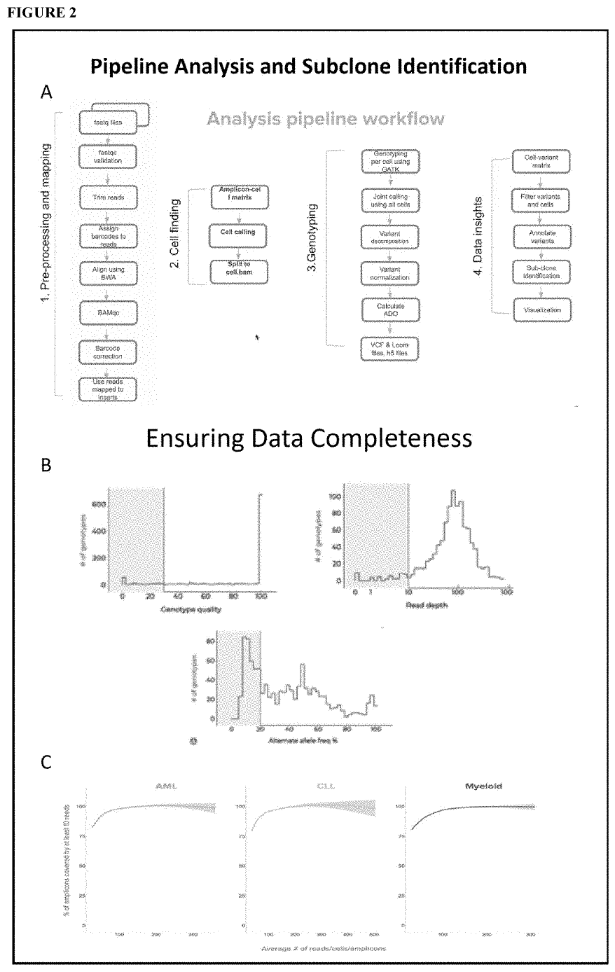

17. A method according to FIG. 2.

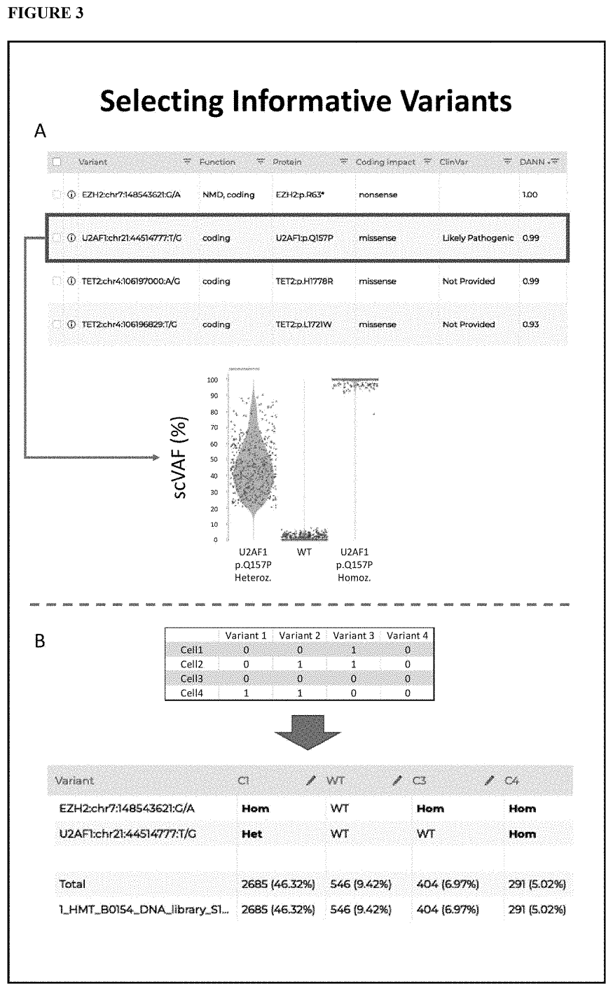

18. A method according to FIG. 3.

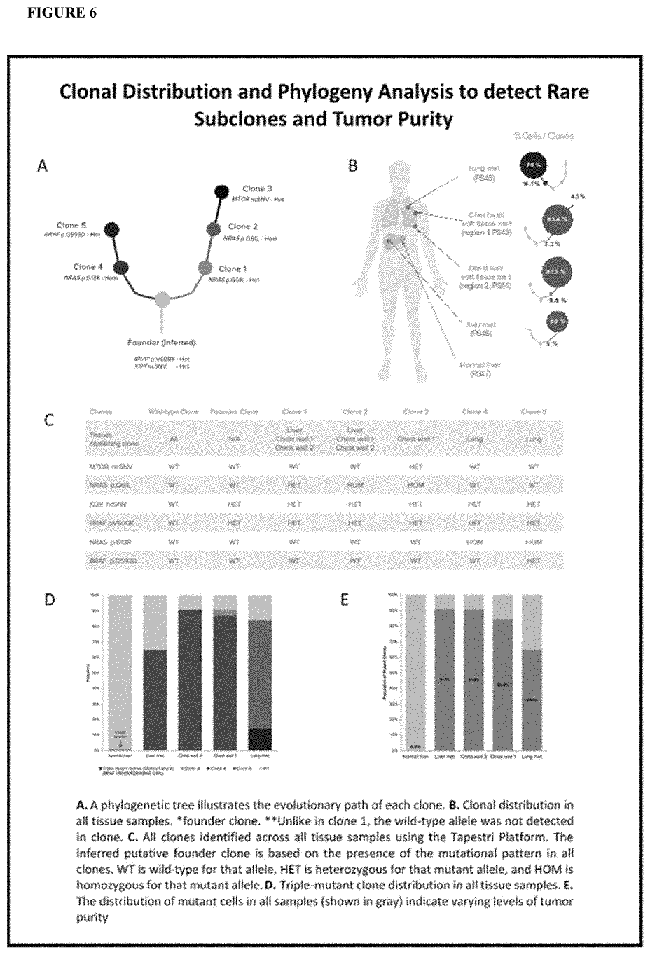

19. A method according to FIG. 6.

20. (canceled)
